(19) United States
(12) Patent Application Publication
(10) Pub. No.: US 2017/0166974 A1
(43) Pub. Date: Jun. 15, 2017

(54) METHOD FOR THE TREATMENT OF MULTIPLE MYELOMA

(71) Applicant: Erasmus University Medical CEnter Rotherdam, Rotterdam (NL)

(72) Inventors: Annemiek Broijl, Rotterdam (NL); Pieter Sonneveld, Rotterdam (NL); Martinus Hendrikus Van Vliet, 's-Gravendeel (NL)

(73) Assignees: Erasmus University Medical Center Rotterdam, Rotterdam (NL); Erasmus University Medical Center Rotterdam, Rotterdam (NL)

(21) Appl. No.: 15/312,544

(22) PCT Filed: May 20, 2014
(86) PCT No.: PCT/EP2014/060357

§ 371 Date: Nov. 18, 2016

(51) Int. Cl. C12Q 1/68; A61K 38/05

(52) U.S. Cl. CPC C12Q 1/6886; A61K 38/05; C12Q 2600/106; C12Q 2600/158; C12Q 2600/118

(57) ABSTRACT

The disclosure is in the field of medical treatments and relates to the treatment of multiple myeloma (MM). In particular, it provides means and methods for the improved treatment of certain subgroups of MM patients, more in particular, patients with a poor prognosis. In a particular embodiment, the disclosure provides a method for determining whether a subject with multiple myeloma is likely to respond to a treatment with a proteasome inhibitor wherein the method comprises the step of performing, on a sample from the subject, a gene expression analysis of a number of N genes selected from the group consisting of the genes NUAK1, ITGB7, AGMAT, TFAP2C, CCDC85A, CLEC7A, TMEM37, RNF144A, and CMPK2, wherein N is at least 2 and wherein it is concluded that the subject is likely to respond to a treatment with a proteasome inhibitor in the case where at least two of the N genes are aberrantly expressed.

### CROSS-REFERENCE TO RELATED APPLICATIONS

This application is a national phase entry under 35 U.S.C. §371 of International Patent Application PCT/EP2014/060357, filed May 20, 2014, designating the United States of America and published in English as International Patent Publication WO 2015/176749 A1 on Nov. 26, 2015.

### TECHNICAL FIELD

The application is in the field of medical treatments and relates to the treatment of multiple myeloma (MM). In particular, it provides means and methods for the improved treatment of certain subgroups of MM patients; more in particular, patients with a poor prognosis. In a particular embodiment, the disclosure provides a method of treatment wherein patients with a poor prognosis are selected and treated with a proteasome inhibitor such as Bortezomib.

### BACKGROUND

Multiple Myeloma (MM) accounts for 10% of all hematological malignancies, with an incidence of five cases per 100,000/year and a median age at onset of 65-70 years. There is a slight male predominance. The incidence of multiple myeloma is twice as high in African Americans as in Caucasian persons. The disease is rarely observed in individuals of Asian descent. It is diagnosed by the presence of monoclonal plasma cell proliferation with more than 10% plasma cells in the bone marrow, presence of monoclonal proteins in serum, and/or in urine with one or more end organ effects such as hypercalcemia, renal failure, anemia, or bone destruction. (Kyle Crab et al., Blood 2008, 111(6):2962-72; Raab et al., Lancet. 2009, 374 (9686):324-39.)

Recent years have seen a dramatic change in the approach toward diagnosing and treating Multiple Myeloma. Newer and more target-specific approaches to treatment have prolonged the survival for patients with multiple myeloma. The survival advantages have been more evident for patients less than 65 years of age, of whom 68% and 53% go on living beyond 5 years and 10 years, respectively (Brenner et al., Haematologica 2009, 94(2):270-5; Painuly and Kumar, Clin. Med Insights Oncol. 2013, 7:53-73).

Treatment regimens have undergone immense changes resulting in significant improvements in treatment tolerability. Additionally, improvements in overall survival have been achieved with newer therapies such as proteasome inhibitors and immunomodulatory drugs (Kumar et al., Blood 2008 111(5):2516-20; Myeloma Trialists' Collaborative Group, J. Clin. Oncol. 1998, 16(12):3832-42).

An important class of novel anti-myeloma drugs interfere with the ubiquitin proteasome system and disrupt the proteolytic machinery of the tumor cells, preferentially enhancing their susceptibility to apoptosis.

However, MM remains an incurable malignancy with a variable overall survival (OS) ranging between a few months to more than 10 years, with 30% surviving 5 years after diagnosis.

Bortezomib, in particular, has shown significant clinical efficacy in myeloma treatment. It is the most commonly used proteasome inhibitor and has been tested to be effective in prolonging the overall survival in several trials (Painuly and Kumar, Clin. Med. Insights Oncol. 2013 7:53-73). Its combinations with cyclophosphamide and dexamethasone are currently among the treatments of choice for MM patients.

Substantial efforts have been made to predict disease outcome in newly diagnosed patients. Prognostic markers, such as serum β2-microglobulin (B2M) and albumin, together constituting the international staging system (ISS), delineate patients into three distinct risk categories (Greipp et al., J. Clin. Oncol. 2005 23:3412-3420).

In addition, MM can be cytogenetically divided into hyperdiploid and nonhyperdiploid MM, with the latter category demonstrating a high proportion of translocations involving the immunoglobulin heavy chain at chromosome 14q32. Together with translocation t(11;14), involving CCND1, hyperdiploid MM has a relatively favorable prognosis as compared to nonhyperdiploid MM. Translocation t(4;14), t(14;16) and t(14;20) and (partial) deletion of chromosome 17 del(17) are considered to be high-risk genetic aberrations.

The University of Arkansas for Medical Sciences (UAMS) generated a molecular classification of myeloma based on gene expression profiles of patients included in their local trials. The UAMS molecular classification of myeloma identifies seven distinct gene expression clusters, including the translocation clusters MS, MF and CD-1/2, a hyperdiploid cluster, a cluster with proliferation-associated genes (PR) and a cluster characterized by a low percentage of bone disease (LB) (Zhan et al., Blood 2006, vol. 108:(6)2020-2028). More recently, this myeloma classification methodology was extended based on the HOVON-65/GMMG-HD4 prospective clinical trial (GSE19784) and additional molecular clusters were identified, that is, NF-κB, CTA and PRL3 (Broyl et al., Blood 2010 116:2543-2553). Because these clusters were discriminated based on disease-specific gene expression profiles, it was hypothesized that they may be relevant for prognosis. Indeed, the UAMS-defined clusters MF, MS and PR were found to identify high-risk disease in the total therapy TT2 trial (Zhan et al., Blood 2006, vol. 108:(6)2020-2028), and patients belonging to the MF, MS, and PR clusters were found to have a poor prognosis.

There remains a need for improved treatment regimes by enabling individual therapy response prediction. This disclosure addresses this need.

### BRIEF SUMMARY

A particular advantageous way of determining whether a subject diagnosed with multiple myeloma (MM) is likely to respond to a treatment with a proteasome inhibitor was found.

In a first aspect, this disclosure provides a method for determining whether a subject suffering from multiple myeloma is likely to respond to a treatment with a proteasome inhibitor, the method comprising the step of performing, on a sample from the subject, a gene expression analysis of a number of N genes selected from the group comprising nine genes according to Table 11, wherein N is at least 2 and wherein it is concluded that the subject is likely to respond to a treatment with a proteasome inhibitor in the case where at least two of the N genes are aberrantly expressed.

In another aspect, this disclosure provides a method for typing a sample from a subject suffering from multiple myeloma as a sample of a subject likely to respond to a treatment with a proteasome inhibitor, the method comprising the step of performing, on the sample, a gene expression analysis of a number of N genes selected from the group comprising nine genes according to Table 11, wherein N is at least 2, and wherein the sample is classified as a sample of a subject likely to respond to a treatment with a proteasome inhibitor in the case where at least two of the N genes are aberrantly expressed in the sample.

Gene expression profiling in aspects of this disclosure is preferably performed by determining the expression level of a selection of genes in an RNA sample. Preferred samples for determining expression levels are samples obtained from tissue, from bone, such as bone marrow or from blood. The sample preferably comprises cancer cells or is suspected to comprise cancer cells.

The disclosure also relates to a method of treating multiple myeloma in a subject, the method comprising: prior to treatment, classifying a subject diagnosed with multiple myeloma as likely to respond to a treatment with a proteasome inhibitor by a method as described above and treating the identified subject with a proteasome inhibitor.

First, the impact of proteasome inhibitors, such as Bortezomib, was evaluated as to survival in relation to cluster designation using the molecular MM clusters as identified in Broyl et al., Blood 2010 116:2543-2553, which reference is incorporated by reference in its entirety herein. In patients treated conventionally, i.e., without proteasome inhibitor, a significant difference was found between all clusters for both overall survival (OS) and progression-free survival (PFS) (p<0.001, for both). The clusters MS, MF and PR demonstrated the shortest survival time, both for OS and PFS.

In the group of Bortezomib-treated patients, those with PR cluster gene expression still demonstrated a poor OS and PFS, but the survival of both MF and MS clusters was clearly improved. Interestingly, also the PFS in groups CD-1, LB and NF-kB improved upon Bortezomib treatment. MM patients classified as belonging to the MF cluster were found to respond best to treatment with a proteasome inhibitor. In particular, the group of MF cluster patients seemed to benefit most from the treatment.

The phrase “MM” or “Multiple Myeloma” is used herein to encompass newly diagnosed or relapse multiple myeloma patients or newly diagnosed Smoldering patients and MGUS (monoclonal gammopathy of undetermined significance) patients.

The phrase “respond to treatment with a proteasome inhibitor” or “benefit from treatment with a proteasome inhibitor” or equivalent as used herein means that a subject either has a longer progression free survival, overall survival or both upon treatment with a proteasome inhibitor compared to an untreated subject or condition or compared to a subject receiving conventional therapy (VAD).

Hence, in a first embodiment, the disclosure relates to a composition comprising a proteasome inhibitor for use in the treatment of a subject suffering from multiple myeloma wherein the subject is classified as belonging to the MF cluster, preferably wherein the subject's classification as an MF cluster patient is based on a gene expression profile of number of N genes selected from the group comprising nine genes according to Table 11, wherein N is at least 2.

In another aspect, the disclosure relates to a method of treating multiple myeloma in a subject, the method comprising performing genetic analysis on a sample from the subject, classifying the subject into a multiple myeloma cluster based on the results of a genetic analysis of a sample from the subject, identifying the subject as having been classified into the MF cluster and treating the identified subject with a proteasome inhibitor, preferably wherein the subject's classification as an MF cluster patient is based on a gene expression profile of number of N genes selected from the group comprising nine genes according to Table 11, wherein N is at least 2.

In yet another aspect, the disclosure relates to a method of treating multiple myeloma in a subject, the method comprising treating the subject with a proteasome inhibitor, wherein the subject has been classified into the MF cluster prior to treatment, preferably wherein the subject's classification as an MF cluster patient is based on a gene expression profile of number of N genes selected from the group comprising nine genes according to Table 11, wherein N is at least 2.

Survival analysis was performed on a group of 301 MM patients, treated with Bortezomib (treated with PAD, a combination of bortezomib, adriamycin, and dexamethasone)) or conventional therapy (treated with VAD, a combination of vincristine, adriamycin, and dexamethasone). The patient group was stratified into ten clusters (Table 1).

TABLE 1
Stratification of patients over 10 MM clusters.

| | Cluster | | | | | | | | | | |
|---|---|---|---|---|---|---|---|---|---|---|---|
| | CD-1 | CD-2 | CTA | HY | LB | MF | MS | Myeloid | NF-kB | PR | Total |
| VAD | 7 | 15 | 12 | 33 | 8 | 8 | 15 | 21 | 22 | 9 | 150 |
| PAD | 6 | 18 | 10 | 44 | 7 | 9 | 18 | 18 | 15 | 6 | 151 |
| Total | 13 | 33 | 22 | 77 | 15 | 17 | 33 | 39 | 37 | 15 | 301 |

In the conventional treatment group (VAD), the MF cluster, consisting of 5% of the patients in this study, demonstrated the shortest median PFS and OS of all the clusters (2 and 4 months, respectively). In marked contrast, in the Bortezomib treatment group, the MF cluster demonstrated a median PFS of 27 months and a median OS of 54 months, which showed the most striking improvement (highest PAD/VAD ratio) in survival from conventional to Bortezomib-based treatment (Tables 2 and 3).

TABLE 2
Progression-free survival of patients in different clusters.

| | Cluster | | | | | | | | | |
|---|---|---|---|---|---|---|---|---|---|---|
| | CD-1 | CD-2 | CTA | HY | LB | MF | MS | Myeloid | NF-κB | PR |
| VAD median PFS [months] | 27 | 41 | 31 | 33 | 33 | 2 | 15 | 36 | 24 | 20 |
| PAD median PFS [months] | 39 | 32 | 31 | 33 | >41 | 27 | 21 | 32 | 32 | 19 |
| PAD/VAD | 1.4 | 0.8 | 1.0 | 1.0 | >1.2 | 13.5 | 1.4 | 0.9 | 1.3 | 1 |

TABLE 3
Overall survival of patients in different clusters.

| | Cluster | | | | | | | | | |
|---|---|---|---|---|---|---|---|---|---|---|
| | CD-1 | CD-2 | CTA | HY | LB | MF | MS | Myeloid | NF-κB | PR |
| VAD | >41 | >41 | >41 | >41 | >41 | 4 | 30 | >41 | >41 | 29 |
| median OS | | | | | | | | | | |
| [months] | | | | | | | | | | |
| PAD | >41 | >41 | >41 | >41 | >41 | 54 | >41 | >41 | >41 | 22 |
| median OS | | | | | | | | | | |
| [months] | | | | | | | | | | |
| PAD/VAD | — | — | — | — | — | 13.5 | >1.4 | — | — | 0.8 |

The median PFS of the MS cluster (10% of studied population) was 15 months in the conventional treatment group, compared to 31 months median survival on average for all other clusters (excluding MS and MF). PFS of the MS cluster was 6 months longer in the Bortezomib treatment group. For OS, the difference was more obvious with a median OS limited to 30 months for conventionally treated patients and median OS not reached (>41 months) for Bortezomib-treated patients.

In the conventionally treated patients, the third cluster with the shortest median PFS, following MF and MS, was the PR cluster with median PFS of 20 months. Whereas both MF and MS demonstrated a clear benefit of Bortezomib treatment, the PR cluster demonstrated a PFS, which is virtually unchanged (19 months). In terms of OS, this cluster showed a median survival of 29 months in conventionally treated patients, whereas the median was 22 months in Bortezomib-treated patients.

The NF-κB cluster demonstrated a median PFS of 24 months in conventionally treated patients compared to 32 months in Bortezomib-treated patients.

Other clusters that demonstrate longer median PFS in Bortezomib-treated patients compared to conventionally treated patients were CD-1 and LB, comprising 4% and 5% of patients, respectively (Tables 2 and 3).

The clusters that demonstrate benefit from Bortezomib treatment include poor prognostic clusters MS and MF, and clusters CD-1, LB and NF-κB. In total, these clusters comprise 36% of this patient population. On the other hand, PR patients (5%) did not demonstrate an improvement on Bortezomib treatment.

It was also found that some patients did even better on conventional treatment than on Bortezomib treatment. Clusters with shorter median PFS after Bortezomib compared to treatment with conventional drugs, included CD-2 (11% of patients, 32 months vs. 41 months, respectively) and Myeloid (12%, 32 months vs. 36 months, respectively).

Finally, two clusters demonstrated no difference in median PFS if treated conventionally or using Bortezomib. These were the CTA cluster and the hyperdiploid cluster (comprising 7% and 24%, respectively).

In conclusion, a clear effect of Bortezomib on the poor prognostic clusters MF and MS was observed, whereas the PR cluster remained a poor prognostic cluster regardless of treatment used. This is graphically represented in Kaplan-Meier plots in FIGS. 1-10.

The disclosure, therefore, relates to a composition comprising a proteasome inhibitor for use in the treatment of a subject with multiple myeloma, wherein the subject belongs to a cluster selected from the group consisting of MS, MF, NF-κB, CD-1 and LB.

More in particular, the disclosure relates to a composition comprising a proteasome inhibitor for use in the treatment of a subject with multiple myeloma wherein the subject belongs to the MF cluster.

The proteasome inhibitor may advantageously be selected from the group consisting of Bortezomib, Carfilzomib, MLN9708, Delanzomib, Oprozomib, Marizomib, AM-114, TMC 95A, Curcusone-D and PI-1840 and combinations thereof. These drugs are also known under different names as shown in Table 4.

TABLE 4
Proteasome inhibitors and their alternative names.

| Drug | Alternative names |
|---|---|
| Bortezomib | VELCADE^{(R)} |
| Carfilzomib | KYPROLIS^{(R)} |
| Ixazomib | MLN9708 |
| Delanzomib | CEP-18770 |
| AM-114 | |
| Oprozomib | ONX 0912 |
| Marizomib | NPI-0052 |
| TMC 95A | |
| Curcusone-D | |
| PI-1840 | |

In preferred embodiments of aspects of this disclosure, the subject belongs to the MF cluster.

The proteasome inhibitor may also be administered in combination with other drugs. In a preferred embodiment, the treatment additionally comprises administering a drug selected from the group consisting of Melphalan, prednisone, doxorubicin, dexamethasone, immunomodulating drugs, monoclonal antibody type drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

In an alternative wording, the disclosure relates to a method of treating multiple myeloma in a subject, the method comprising: performing genetic analysis on a sample from the subject; classifying the subject into a multiple myeloma cluster based on the results of a genetic analysis of a sample from the subject; identifying the subject as having been classified into a cluster selected from the group consisting of MS, MF, CD-1, LB, and NF-κB; and treating the identified subject with a proteasome inhibitor.

In a preferred embodiment, the disclosure relates to a method of treating multiple myeloma in a subject, the method comprising: performing genetic analysis on a sample from the subject; classifying the subject into a multiple myeloma cluster based on the results of a genetic analysis of a sample from the subject; identifying the subject as having been classified into the MF cluster and treating the identified subject with a proteasome inhibitor.

In yet another preferred embodiment, the disclosure relates to a method as described above, wherein the subject undergoes autologous and/or allogenic stem-cell rescue and/or wherein the subject is human.

In yet another alternative wording, the disclosure relates to a method of treating multiple myeloma in a subject, the method comprising: treating the subject with Bortezomib, wherein the subject has been classified into a multiple myeloma cluster selected from the group consisting of MS, MF, CD-1, LB, and NF-κB prior to treatment.

In a preferred embodiment, the disclosure relates to a method of treating multiple myeloma in a subject, the method comprising: treating the subject with Bortezomib, wherein the subject has been classified into the MF cluster prior to treatment.

Gene expression analysis was found to be an advantageous way of clustering of MM patients. The disclosure, therefore, relates to a method as described above, wherein the genetic analysis is a gene expression analysis. Particularly good results were obtained when the gene expression analysis was a microarray analysis. Alternative means for gene expression analysis may, however, be equally well suited, such as, but not limited to, gene expression analysis methods selected from the group consisting of gene array analysis, sequencing of RNA, RNA-FISH, quantitative-PCR, Northern Blotting, Multiplex Ligation-Dependent Probe Amplification and PCR.

In studies with a large number of patients, it has been described that patients may be classified into a particular cluster based on gene expression analysis (Broyl et al., Blood 2010). However, there have hitherto been no methods available for reliably assigning a single patient to one of the known MM clusters.

A particularly advantageous method for classifying an individual subject into one of the MM clusters that employs gene expression profiling using expression profiles of a limited number of genes is described herein. Preferably, the method employs gene array technology. In highly preferred embodiments of aspects of this disclosure, the gene expression level is determined using the probesets of any one of Tables 5-11. In these tables, the indication “Probeset ID” corresponds to the Affymetrix (Santa Clara, Calif.) identifier from the Human Genome U133 Plus2.0 microarray chip set oligonucleotide arrays as described in the Examples below. These identifiers indicate probesets with which one or more unique gene transcripts are identified. The term “gene” in the context of Tables 5-11 as disclosed herein, therefore, include reference to gene transcripts. Preferably, in aspects of this disclosure, the gene expression level of at least two genes selected from the group comprising the top 100 genes for each cluster as shown in Table 10 is determined. A particular patient may, for instance, be assigned to the MF cluster by determining the expression of at least two genes selected from the group consisting of the top 100 genes of the MF cluster as shown in Table 10. Any combination of two genes selected from the group of genes listed for the MF cluster in Table 10 was sufficient to allocate the patient to that particular cluster. The same was found to be true for the other clusters in Table 10.

In even more advantageous embodiments of aspects of this disclosure, the gene expression analysis includes the step of determining the expression profile of at least two genes selected from the group consisting of genes indicated in Table 10.

In yet another advantageous embodiment, the disclosure, therefore, relates to methods and aspects as described above, wherein the gene expression analysis includes the expression profile of at least the first two genes of Table 10 for each of the clusters MF, MS, NF-κB, and LB.

Preferred aspects of this disclosure include the step of determining the expression of more than two genes. This includes the expression of 3, 4, 5, 6, 7, 8, 9, 10, 11, 12, 13, 14, 15, 16, 17, 18, 19, 20, 21, 22, 23, 24, or more genes.

An optimal number of genes appeared to be 20 genes for the MS cluster (Table 5), nine genes for the MF cluster (Table 6 and Table 11), 24 genes for the CD-1 cluster (Table 7), 21 genes for the NF-κB cluster (Table 8) and five genes for the LB cluster (Table 9).

The term “subject with multiple myeloma” or “MM subject” refers to a subject, or patient, that has been diagnosed as having multiple myeloma. Results of any single test are generally not enough to diagnose multiple myeloma. Diagnosis is based on a combination of factors, including the patient's description of symptoms, the doctor's physical examination of the patient, and the results of blood tests and optional x-rays. The diagnosis of multiple myeloma in a subject may occur through any established diagnostic procedure known in the art. Generally, multiple myeloma is diagnosed when a plasma cell tumor is established by biopsy, or when at least 10% of the cells in the bone marrow are plasma cells in combination with the finding that either blood or urine levels of M protein are over a certain level (e.g., 3 g/dL and 1 g/dL, respectively) or holes in bones due to tumor growth or weak bones (osteoporosis) are found on imaging studies.

Without wishing to be bound by theory, it is put forward herein that the proteasome inhibitor for use as described herein exerts its function through its interaction with the 26S proteasome. The 26S proteasome is an essential protein complex that regulates protein degradation and protein re-localization in all cells including cancerous cells. It is involved in many cellular processes including proliferation, apoptosis, and degradation of misfolded proteins. Furthermore, the proteasome plays a critical role in the degradation of disease-related proteins. The proteasome recognizes the ubiquitin molecule tag, which is attached to proteins by a three-step ubiquitination process.

Proteins that are targeted for degradation and re-localization are marked by a ubiquitin chain, which is recognized by the proteasome. Dependent on the localization of the ubiquitin, the protein will be processed differently by the proteasome. Proteins tagged with lysine 48-linked ubiquitin chains are marked for degradation. Proteins that are tagged with a single ubiquitin group or with lysine 63-linked chains of ubiquitin are marked for alternative biological processes including re-localization.

Degradation of protein substrates by the proteasome requires the protein to traverse the regulatory gate (19S) of the proteasome and interact with the proteolytic enzymes in the catalytic core (20S). The catalytic core particle of the proteasome forms the protein degradation machinery of the proteasome. Poly-ubiquitinated proteins (substrates) are processed in the catalytic core particle of the proteasome. The proteasome complex is currently commonly referred to as the 26S proteasome. Following gate opening, substrates translocate into the catalytic chamber of the core particle, where several active degradation sites exist.

Inhibition of the proteasome is a unique approach in cancer treatment. Preclinical activity is shown in many tumor types including solid tumors. The potential use of proteasome inhibitors in cancer treatment has been extensively described in Adams et al., Cancer Research 59:2615-2699 (1999) [18]. Current proteasome inhibitors bind to, and influence the catalytic core particle of the proteasome. Bortezomib or PS-341 was the first proteasome inhibitor that received FDA approval. Nowadays, other proteasome-targeted treatments are in different stages of development for application in various diseases including, but not limited to, cancer.

Although the exact down-stream mechanism by which proteasome inhibitors lead to cell death of malignant cells in vitro and in vivo has not yet been fully elucidated, studies indicate that proteasome inhibitor-induced malignant cell death is associated with induction of the endoplasm reticulum, stress and activation of the unfolded protein response, inhibition of the NF-κB inflammatory pathway, activation of caspase-8 and apoptosis, and increased generation of reactive oxygen species.

The positive effect in cancer is most likely the result of the inhibition of proteasome-regulated degradation and, therefore, accumulation of (pro-apoptotic) proteins. In addition, studies have shown that proteasome inhibitors are selective for cancer cells. Cancer cells appear to have an increased sensitivity for proteasome inhibitors, a similar effect is observed in chemotherapies.

Interfering with the 26S proteasome forms a unique approach in cancer treatment. In itself, the proteasome is a highly conserved protein complex. Furthermore, the proteasome is a relatively independent protein complex that can be described as a highly regulated trash bin mechanism for efficient protein management in all cells of the human body. As a result, downstream effects of proteasome inhibition are similar. Proteasome inhibitors inhibit the degradation machinery, followed by accumulation of proteins, which drives the elimination of tumor cells. Therefore, it is likely that a patient who would benefit from the positive effects of Bortezomib treatment would also benefit from the positive effects of an alternative proteasome inhibitor.

In a preferred embodiment of the disclosure, the proteasome inhibitor is Bortezomib. Bortezomib reversibly blocks the function of the proteasome of the cell, affecting numerous biologic pathways, including those related to growth and survival of cancer cells. However, the disclosure also relates to a composition for a use or method as described herein wherein the proteasome inhibitor is selected from the group consisting of Bortezomib, Carfilzomib, MLN9708, Delanzomib, Oprozomib, AM-114, Marizomib, TMC-95A, Curcusone-D and PI-1840.

Currently, Bortezomib has been approved for use in patients with multiple myeloma, who have already received at least one prior treatment and whose disease is worsening on their last treatment and who have already undergone or are unsuitable for bone marrow transplantation. Bortezomib has significant activity in patients with relapsed multiple myeloma and MM patients that suffer from renal insufficiency.

The efficacy or outcome of the treatment with Bortezomib is known to increase when Bortezomib is used in combination with dexamethasone. Its efficacy has even been shown to be improved in a synergistic way when used in combination with other drugs, such as doxorubicin.

Proteasome inhibitors may, therefore, be used in aspects of the disclosure, either alone or in combination with other drugs, such as drugs selected from the group consisting of Melphalan, prednisone, doxorubicin, dexamethasone, immunomodulating drugs, monoclonal antibody drugs, including drugs based on antibody fragments, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

The use of Bortezomib in combination with at least one drug selected from the group consisting of Melphalan, prednisone, doxorubicin, dexamethasone, immunomodulating drugs, monoclonal antibody drugs, including drugs based on antibody fragments, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors is preferred.

The compositions for use as described herein or the methods of treatment as described herein has several advantages over prior art treatments of multiple myeloma. In the prior art treatments, Bortezomib was administered to MM patients without the pre-selection whether or not the patient belonged to the MF cluster or had, for instance, an aberrant expression of at least two genes selected from the nine genes according to Table 11. This resulted in the over-treatment of subjects that may not benefit from a treatment with proteasome inhibitors.

The term “aberrant expression” or “aberrantly expressed” refers to overexpression or underexpression of a given gene. Over-expression occurs if the expression of a gene is higher than a reference level; under-expression occurs when the expression level of a gene is below a reference level. The reference level may be arbitrarily chosen or empirically determined. In a preferred embodiment, the reference level is a normal expression level, i.e., the expression level of a normal, healthy, control subject. In another preferred embodiment, the reference level is the average expression level of the gene in a population of control subjects. For the purpose of determining whether a gene is aberrantly expressed in an MM patient, the reference expression level is advantageously the expression level of the gene in a control MM patient or a population of MM patients. For example, Table 6 and Table 11 show that genes CCDC85A, RNF144A and CMPK2 are under-expressed, whereas genes NUAK1, ITGB7, AGMAT, TFAP2C, CLEC7A and TMEM37 are over-expressed in MM patients belonging to the MF cluster or likely to respond to a treatment with a proteasome inhibitor. Over-expression and under-expression in Table 11 are determined using the average expression of the respective gene in a population of MM patients as the reference value.

Table 6 shows the eleven probe sets used for determining aberrant expression of nine genes as indicated using gene chip array technology. Equivalent or the same results may be obtained when other methods of determining gene expression are used. These other methods may include different probe sets or even entirely different technology. It is an aspect of this disclosure that as long as the expression of two genes selected from the group of nine genes of Table 6 or Table 11 is used, methods employed in aspects of this disclosure provide reliable and accurate results for allocating a subject to the MF cluster of MM patients.

Proteasome inhibitors may cause severe peripheral neuropathy, causing pain and (severe) physical disabilities as a result, with some patients even ending up in wheel chairs. Additionally, the proteasome inhibitors may be administered intravenously or subcutaneously, which can cause very high toxic doses at the site of administration. This route of administration also requires the patients to travel to a physician, which, in many cases can be a serious limitation because these patients can be in poor condition and/or live far from their physicians.

The use of proteasome inhibitors is, therefore, preferably prevented in patients that will receive little or no benefit from the treatment compared to other available treatments. As indicated herein above, MM patients belonging to the MS, MF, CD-1, LB and NF-κB clusters exhibit either longer progression-free survival, overall survival, or both, upon treatment with a proteasome inhibitor. As indicated herein above, MM patients not belonging to either of the clusters MS, MF, CD-1, LB and NF-κB, but instead belonging to the CD-2, CTA, HY, Myeloid and PR clusters, either do not benefit in the sense of exhibiting longer progression-free survival or overall survival upon treatment with a proteasome inhibitor, or even show adverse response in the progression-free survival or overall survival decrease upon treatment with a proteasome inhibitor.

The disclosure, therefore, also relates to a method of treating MM in a subject, the method comprising administering to the subject a treatment regime that does not comprise a proteasome inhibitor, wherein the subject has previously been diagnosed as belonging to the CD-2, CTA, HY, Myeloid or PR cluster. Whether an MM patient belongs to the CD-2, CTA, HY, Myeloid or PR cluster may, for instance, be determined by establishing that the MM patient does not belong to any of the clusters MS, MF, CD-1, NF-κB and LB. This may advantageously be achieved by determining gene expression levels in the patient using either the negative (non-cluster)-classifiers or the positive (cluster) classifiers indicated in Tables 5-9, for each of these clusters, respectively, and showing that on the basis of at least two genes, the patient does not have an aberrant gene expression level for any of the clusters MS, MF, CD-1, NF-κB or LB. For instance, a non-MF cluster subject does not exhibit an aberrant expression of at least two genes selected from the nine genes according to Table 6 or Table 11.

In a preferred embodiment, the disclosure relates to a method as described above, wherein the administration of the proteasome inhibitor to the subject is made with the knowledge that the proteasome inhibitor is less effective in the treatment of patients that do not belong to the MF cluster or that do not exhibit an aberrant expression of at least two genes selected from the nine genes according to Table 11.

When applying a method according to this disclosure, patients that benefit most from the treatment (responders) may be selected and separated from patients that are less likely to benefit from the treatment (non-responders), which translates into a significant decrease of (unnecessary) proteasome inhibitor treatment and, consequently, fewer patients suffer from adverse events.

The method of treatment according to the disclosure thus leads to cost reduction by preventing the use of unnecessary expensive treatment, and preventing unnecessary follow-up and hospitalization of patients on (serious) adverse events.

In a preferred aspect, the disclosure relates to a method of treating a subject with MM, the method comprising subjecting a subject with MM to a treatment regime that comprises the administration of a proteasome inhibitor, wherein the subject prior to treatment has been diagnosed belonging to the MF cluster or had an aberrant expression of at least two genes selected from the nine genes according to Table 11, wherein the treatment optionally further comprises the administration of at least one drug selected from the group consisting of Melphalan, prednisone, doxorubicin, dexamethasone, immunomodulating drugs, monoclonal antibody drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, insulin-like growth factor inhibitors, RAS inhibitors, PARP-inhibitors and B-RAF inhibitors.

A new way of determining whether a subject with multiple myeloma belongs to the MF cluster or is likely to respond to a treatment with a proteasome inhibitor was also discovered. For that, a method was provided based on gene expression analysis. Table 11 provides a gene set for use in determining whether a subject with MM belongs to MF cluster or is likely to respond to a treatment with a proteasome inhibitor. The abbreviations of the genes (Gene Symbol) and the probe set are sufficient for a skilled person to unequivocally determine the relevant genes. Details may be obtained from the World Wide Web at affymetrix.com/support/technical/annotationfilesmain.affx. Details of the database are as follows: Affymetrix, netaffx-annotation-date=2012-10-15, netaffx-annotation-netaffx-build=33, genome-version=hg19, genome-version-ncbi=GRCh37.

It was found that individuals that belong to the MF cluster or individuals that are likely to respond to a treatment with a proteasome inhibitor could be distinguished from other subjects with MM by determining the normalized expression level of at least two genes selected from the group of nine genes provided in Table 11, wherein the subject belongs to the MF cluster or is likely to respond to a treatment with a proteasome inhibitor if at least two genes were aberrantly expressed.

Hence, in highly preferred embodiments of aspects of this disclosure, the normalized expression level of at least two genes is determined selected from the group of nine genes provided in Table 11, wherein the subject belongs to the MF cluster or is likely to respond to a treatment with a proteasome inhibitor if at least two of the genes, preferably 3, 4, 5, 6, 7, 8 or 9 genes, are aberrantly expressed.

Determining expression levels of genes in aspects of this disclosure preferably comprises the performance of gene expression analysis on samples of a subject, preferably nucleic acid samples, such as nucleic acid samples obtained after isolating nucleic acids from bone, tissue or fluid samples of a subject with MM. Methods for performing of gene expression analysis on samples are well known in the art.

As used herein, the term “nucleic acid samples” refers to samples obtained from a subject that contain nucleic acids, such as samples obtained from bone, blood or tissue, preferably from plasma cells.

As used herein, the term “normalized expression level” means the expression level of a gene of interest (selected from the group of nine genes of Table 11) divided by a reference expression level. This reference expression level or reference expression value may be arbitrarily chosen but is preferably the expression level of the gene of interest as determined in at least one control individual diagnosed with MM. Even more preferred, the reference level is the expression level of the gene of interest in a control individual diagnosed with MM that does not belong to the MF group. Most preferred is a reference expression level derived from a group of control individuals such as the ones described above. Such a preferred reference value may be derived by calculating the average expression level from a group of control individuals diagnosed with MM that do not belong to the MF group.

The expression levels of the genes according to Table 11 may be determined in RNA samples obtained from plasma cells, wherein CD138, CD319 or CD269 surface protein-positive cells are preferred.

The term “over-expressed” is used herein to indicate a level of expression that is above a reference expression level. The skilled person is familiar with methods for determining reference expression levels. In a preferred embodiment, the expression level determined in the method according to the disclosure is at least 10% above the reference value, such as 20%, 30%, 40%, 50%, 60%, 70%, 80%, 90% or even more than 100% above the reference value such as 100, 200, 300 or even 400% or more above the reference value.

The term “under-expressed” is used herein to indicate a level of expression that is below a reference expression level. The skilled person is familiar with methods for determining reference expression levels. In a preferred embodiment, the expression level determined in the method according to the disclosure is at least 10% below the reference value, such as 20%, 30%, 40%, 50%, 60%, 70%, 80%, 90% or even more than 100% below the reference value such as 100%, 200%, 300% or even 400% or more below the reference value.

The group of genes presented in Table 11 may, therefore, be used to determine whether a subject with MM belongs to the MF cluster or is likely to respond to a treatment with a proteasome inhibitor or not. The expression level of any set of two genes selected from Table 11 may be determined and compared to a reference expression level for the particular gene set. If the expression level of each of the two genes is aberrant, then the subject belongs to the MF cluster or is likely to respond to a treatment with a proteasome inhibitor.

There are a great number of suitable techniques known in the art for determining expression levels of genes. Those include, but are not limited to, gene expression array analysis, (Next generation) sequencing of RNA, RNA-FISH, quantitative-PCR, Northern Blotting, MLPA, microarray GEP, PCR, and others.

The method may even be improved by determining the expression level of more than two genes such as 3, 4, 5, 6, 7, 8, or 9 genes selected from Table 11.

In machine learning and statistics, classification is the problem of identifying to which of a set of categories a new observation belongs, on the basis of a training set of data containing observations (or instances) whose category membership is known. An algorithm that implements classification, especially in a concrete implementation, is known as a classifier.

Many classifiers are known in the art, with linear or non-linear classifier boundaries, such as, but not limited to: ClaNC, nearest mean classifier, simple Bayes classifier, linear discriminant analysis (LDA), quadratic discriminant analysis (QDA), Support Vector Machines (SVM), or the k-nearest neighbor (k-nn) classifier.

In a particularly advantageous embodiment, the disclosure relates to a method that includes a linear classifier. The ClaNC classifier (Classification to Nearest Centroids) is such a linear classifier. In that classifier, for a single MM patient called x, a distance d to each of the two centroids is calculated. Centroids are referred to with 0 and 1 subscripts here (wherein 1 reflects patients likely to respond to a treatment with a proteasome inhibitor and wherein 0 reflects patients likely not to respond to a treatment with a proteasome inhibitor). The employed distance is the normalized Euclidean distance measure, resulting in a d₀ and a d₁, formulated as:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$

wherein x₁ represents the expression level of a particular gene i of the subject x, wherein gene i is chosen from the group comprising nine genes according to Table 11, wherein N is the total number of genes selected from the group comprising nine genes according to Table 11, wherein m₀ and s₀ are values according to Table 11, wherein mᵢ is the mean of the centroid for gene i according to Table 11, and wherein sᵢ is the standard deviation of the centroid for gene i according to Table 11.

The MM patient is then assigned to the group with the smallest distance d (i.e., the closest centroid). It is, therefore, concluded that the subject x is likely to respond to treatment with a proteasome inhibitor if the value for d₁ is less than the value for d₀ or wherein it is concluded that the subject x is likely not to respond to a treatment with a proteasome inhibitor if the value for d₀ is less than or equal to the value for d₁.

An example of a determination according to a preferred embodiment of the disclosure is provided in Example 4.

The teaching as provided herein should not be interpreted so narrowly that the exact values as provided in Table 11 are the only way of arriving at the desired result. While providing the best mode of performing the disclosure when used as provided in Table 11, the numbers for m₀, m₁, s₀ and s₁ may be used as a guideline, in such a way that values that are 50% above or below these numbers will still yield satisfactory results. It should be noted in this respect that increasingly more accurate and reliable results may be obtained when the values for m₀, m₁, s₀ and s₁ resemble the values as provided in Table 1. In that respect, values that are only 10% different will provide better results than values that are 20, 30 or 40% different from the values provided in Table 1.

In an alternative embodiment, the numbers may be rounded off to 1 or 2 decimals without departing from the spirit of the disclosure.

In summary, the disclosure relates to a method for determining whether a subject diagnosed with multiple myeloma is likely to respond to a treatment with a proteasome inhibitor wherein the method encompasses the step of performing, on a sample from the subject, a gene expression analysis of a number of N genes selected from the group comprising nine genes according to Table 11, wherein N is at least two and wherein it is concluded that the subject is likely to respond to a treatment with a proteasome inhibitor in case that at least two of the N genes are aberrantly expressed.

The disclosure also relates to a method as described above, comprising the steps of:
a. providing at least one probe for the detection of the expression level of N genes selected from the group comprising nine genes according to Table 11,
b. contacting the probe with a sample comprising mRNA originating from a patient, and
c. determining the expression level of each individual gene from the at least N genes.

The term “probe” refers to an oligonucleotide consisting of RNA or DNA capable of specifically hybridizing to the gene of interest. A skilled person is well aware of the metes and bounds for the effective design of a probe. A single probe may be sufficient for detection of gene expression, for instance, by a gene array analysis. In an advantageous embodiment, the at least one probe comprises a probe set, i.e., two probes capable of hybridizing in forward and reverse orientation at opposite ends of a nucleotide region to be amplified. Such may be advantageous in PCR analysis or sequencing.

The method as described above may be improved by using more than two genes selected from Table 11 in the gene expression analysis. In an advantageous embodiment, the method as described above employs N genes wherein N is at least 3, 4, 5, 6, 7, 8, or at least 9.

The conclusion that a subject belongs to the MF cluster or is likely to respond to a treatment with a proteasome inhibitor may be based on the aberrant expression level of two genes as described above. This may be further improved when the conclusion is based on the expression level of between two and N genes.

Other means of gene expression analysis are equally well suited. Non-limiting examples of such techniques include: gene array analysis, sequencing of RNA, RNA-FISH, quantitative-PCR, Northern Blotting, Multiplex Ligation-Dependent Probe Amplification, microarray gene expression profiling and PCR. The use of a gene expression chip is, however, preferred.

Patients identified with a method as described herein may advantageously be treated with a proteasome inhibitor selected from the group consisting of Bortezomib, Carfilzomib, MLN9708, Delanzomib, Oprozomib, AM-114, Marizomib TMC-95A, Curcusone-D and PI-1840. Use of Bortezomib is preferred.

In addition to the proteasome inhibitor, selected patients may be treated with a drug selected from the group consisting of Melphalan, prednisone, doxorubicin, dexamethasone, immunomodulating drugs, monoclonal antibody type drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

Advantageously, the gene expression analysis is performed on a sample comprising plasma cells.

In order to determine whether an aberrant gene expression is indicative that a subject belongs to the MF cluster or is likely to respond to a treatment with a proteasome inhibitor, a classifier such as a linear classifier may advantageously be employed.

A particularly preferred classifier is a ClaNC (Classification to Nearest Centroids) classifier. Therein, for a single subject x with multiple myeloma, a distance d₀ and d₁ is calculated, wherein d₀ and d₁ are defined by the formulas 1 and 2:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$

wherein xᵢ represents the expression level of a particular gene i of the subject x, wherein gene i is chosen from the group comprising nine genes according to Table 11, wherein N is the total number of genes selected from the group comprising nine genes according to Table 11, wherein m₀ and s₀ are values according to Table 11, wherein mᵢ is the mean of the centroid for gene i according to Table 11, and wherein sᵢ is the standard deviation of the centroid for gene i according to Table 11 and wherein it is concluded that the subject x is likely to respond to treatment with a proteasome inhibitor if the value for d₁ is less than the value for d₀ or wherein it is concluded that the subject x is likely not to respond to a treatment with a proteasome inhibitor if the value for d₀ is less than or equal to the value for d₁.

The disclosure also relates to a method of treating multiple myeloma in a subject, the method comprising:
a) prior to treatment, classifying a subject diagnosed with multiple myeloma as likely to respond to a treatment with a proteasome inhibitor in a method as described above; and
b) treating the identified subject with a proteasome inhibitor.

In a preferred embodiment, the disclosure relates to a method as described above wherein the proteasome inhibitor is selected from the group consisting of Bortezomib, Carfilzomib, MLN9708, Delanzomib, Oprozomib, AM-114, Marizomib, TMC-95A, Curcusone-D and PI-1840. The proteasome inhibitor is preferably Bortezomib.

In addition, the treatment preferably comprises a drug selected from the group consisting of Melphalan, prednisone, doxorubicin, dexamethasone, immunomodulating drugs, monoclonal antibody type drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

In other terms, the disclosure relates to a composition comprising a proteasome inhibitor for use in the treatment of a subject with multiple myeloma wherein the subject has been diagnosed, prior to treatment, as likely to respond to a treatment with a proteasome inhibitor in a method as described herein.

The method as described above may also be used to determine whether a subject x, diagnosed with multiple myeloma belongs to the MF cluster. Such a method calculates the distances d₀ and d₁ to each of the two centroids, defined by the formulas 1 and 2:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$

wherein xᵢ represents the expression level of a particular gene i of the subject x, wherein gene i is chosen from the group comprising nine genes according to Table 11, wherein N is the total number of genes selected from the group comprising nine genes according to Table 11, wherein m₀ and s₀ are values according to Table 11, wherein mᵢ is the mean of the centroid for gene i according to Table 11, and wherein sᵢ is the standard deviation of the centroid for gene i according to Table 11 and wherein it is concluded that the subject is likely to respond to treatment with a proteasome inhibitor if the value for d₁ is less than the value for d₀ or wherein it is concluded that the subject x is likely to belong to the MF-cluster if the value for d₁ is less than the value for d₀ or that the subject x is likely not to belong to the MF-cluster if the value for d₀ is less than or equal to the value for d₁.

### BRIEF DESCRIPTION OF THE DRAWINGS

FIG. 1. Kaplan Meier curves for the MS cluster showing cumulative Progression-Free Survival versus time in months. Dashed black line is PAD-treated group; solid grey line is the VAD-treated group.

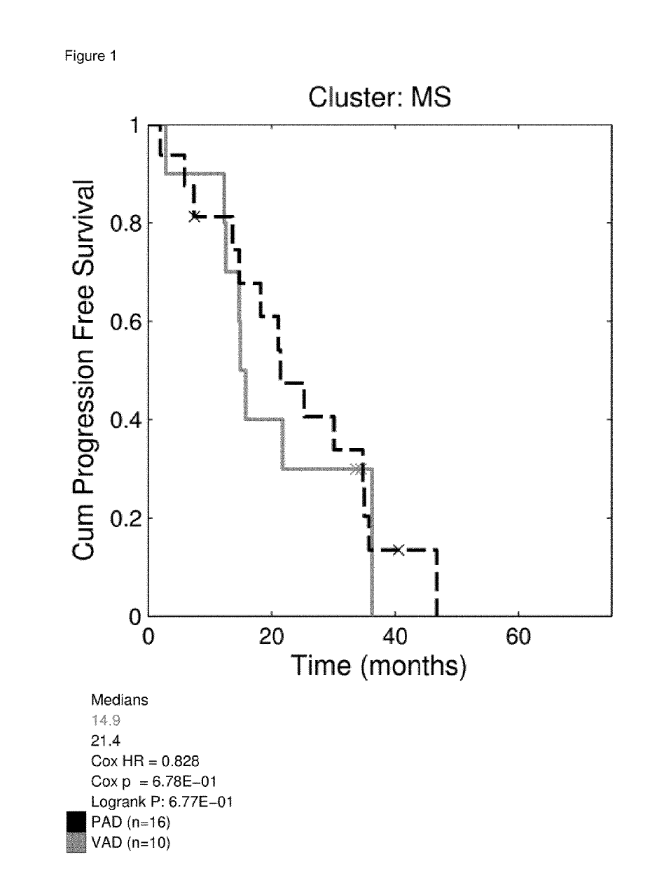

FIG. 2. Kaplan Meier curves for the MF cluster showing cumulative Progression-Free Survival versus time in months. Dashed black line is PAD-treated group; solid grey line is the VAD-treated group.

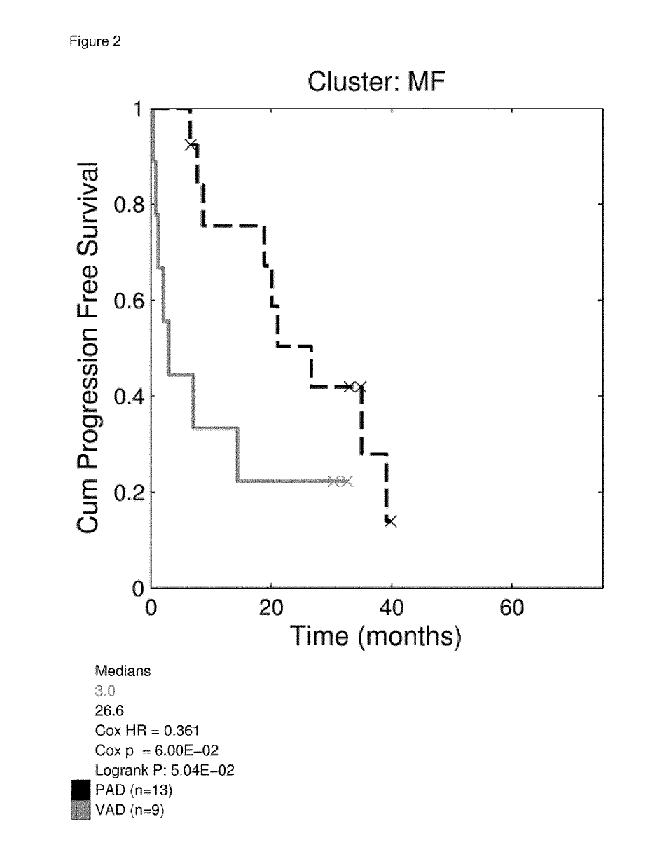

FIG. 3. Kaplan Meier curves for the CD-1 cluster showing cumulative Progression-Free Survival versus time in months. Dashed black line is PAD-treated group; solid grey line is the VAD-treated group.

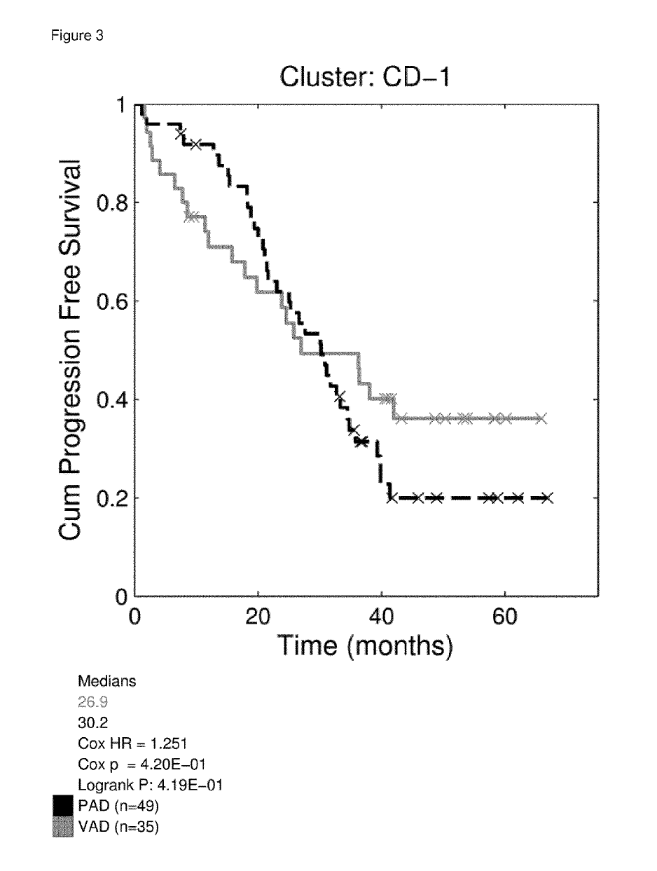

FIG. 4. Kaplan Meier curves for the NF-kB cluster showing cumulative Progression-Free Survival versus time in months. Dashed black line is PAD-treated group; solid grey line is the VAD-treated group.

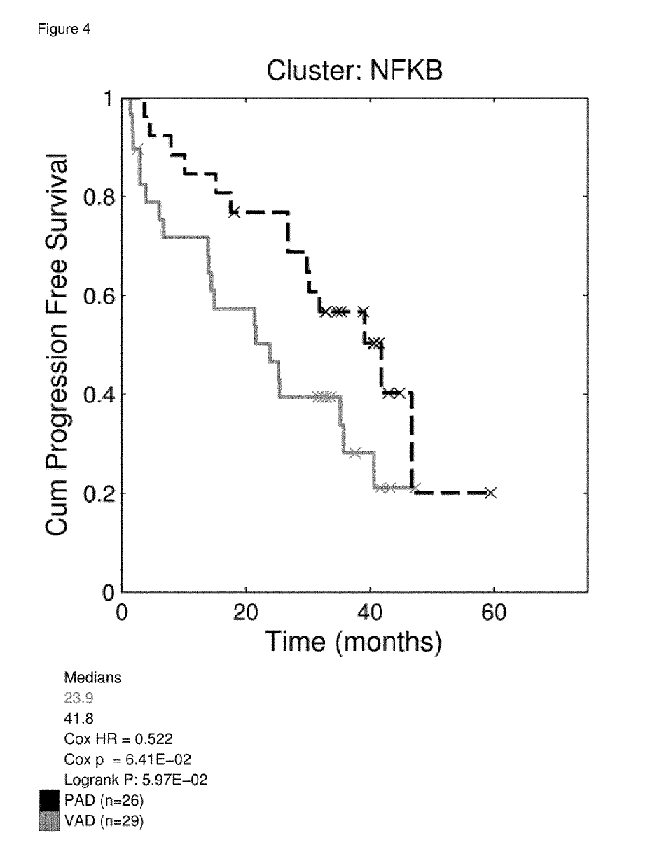

FIG. 5. Kaplan Meier curves for the LB cluster showing cumulative Progression-Free Survival versus time in months. Dashed black line is PAD-treated group; solid grey line is the VAD-treated group.

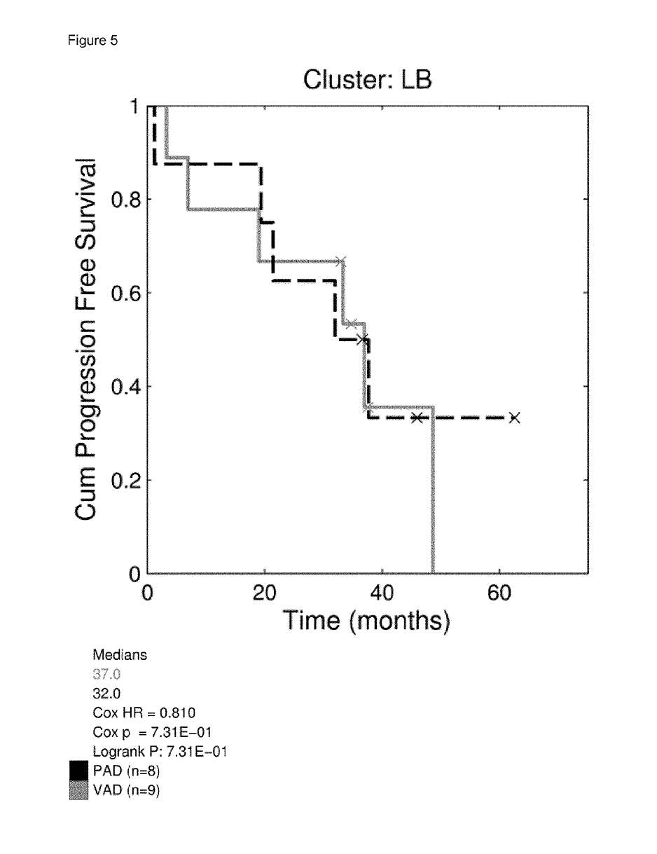

FIG. 6. Kaplan Meier curves for the MS cluster showing cumulative Overall Survival versus time in months. Dashed black line is PAD-treated group; solid grey line is the VAD-treated group.

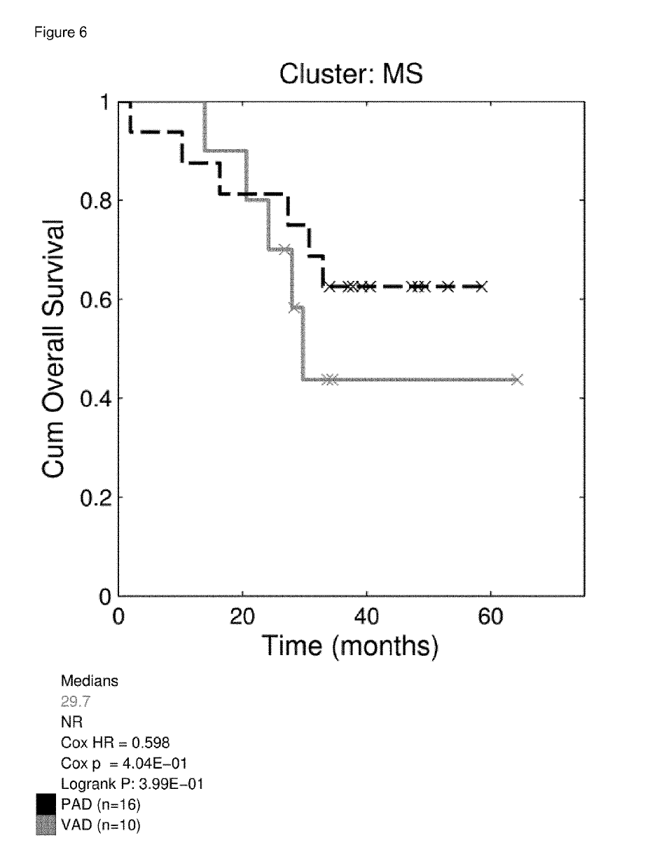

FIG. 7. Kaplan Meier curves for the MF cluster showing cumulative Overall Survival versus time in months. Dashed black line is PAD-treated group; solid grey line is the VAD-treated group.

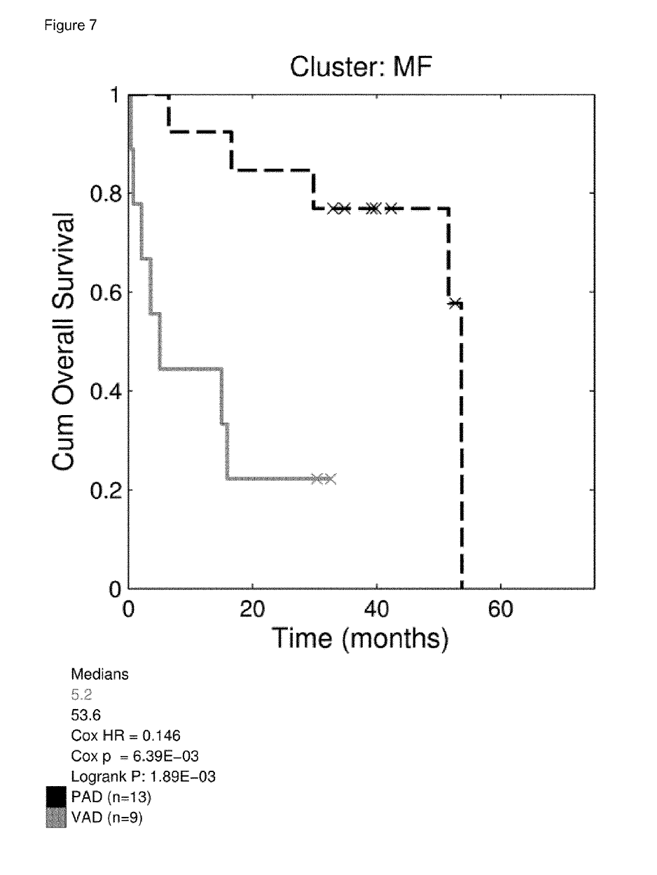

FIG. 8. Kaplan Meier curves for the CD-1 cluster showing cumulative Overall Survival versus time in months. Dashed black line is PAD-treated group; solid grey line is the VAD-treated group.

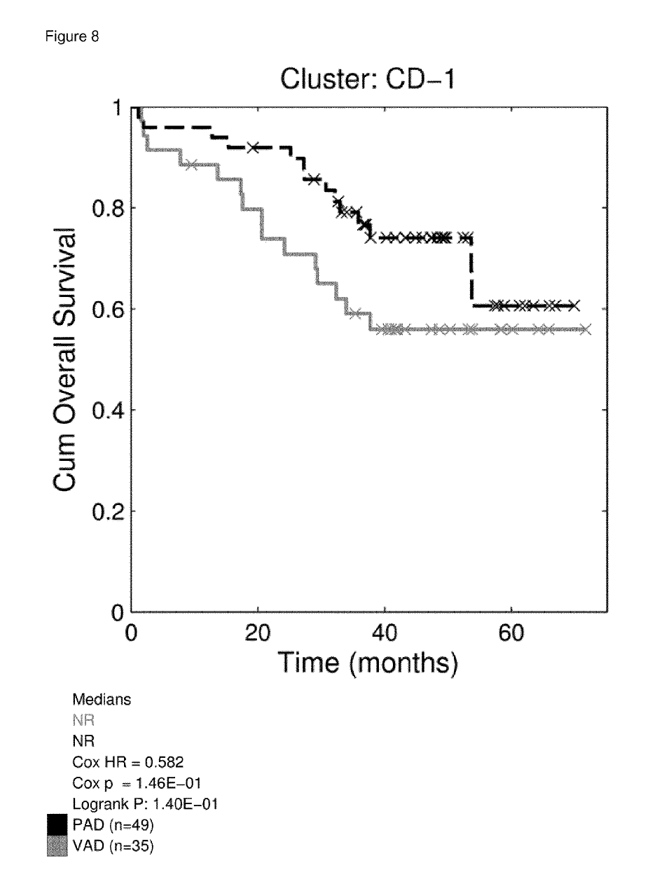

FIG. 9. Kaplan Meier curves for the NF-kB cluster showing cumulative Overall Survival versus time in months. Dashed black line is PAD-treated group; solid grey line is the VAD-treated group.

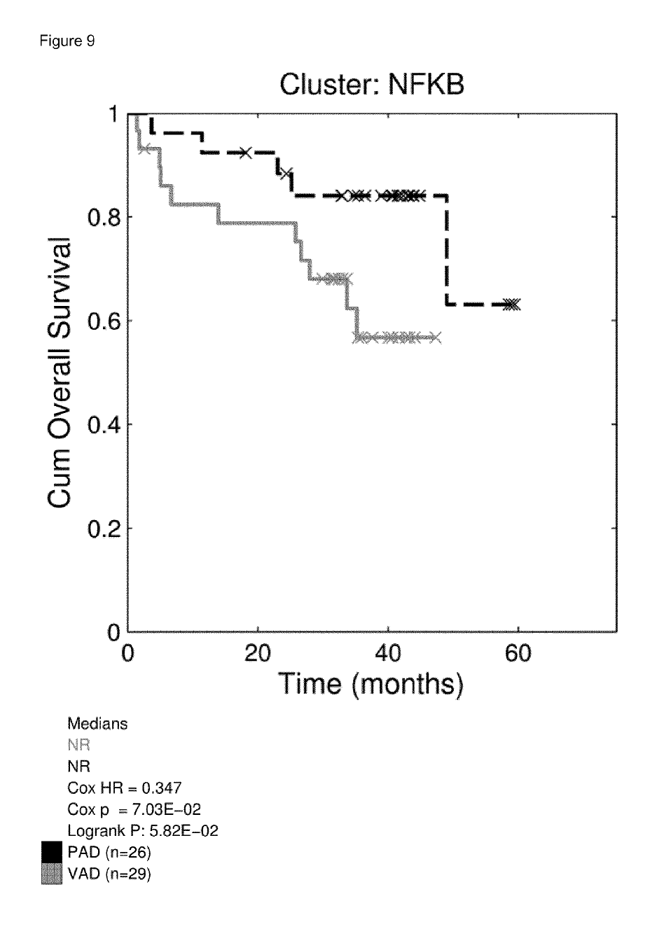

FIG. 10. Kaplan Meier curves for the LB cluster showing cumulative Overall Survival versus time in months. Dashed black line is PAD-treated group; solid grey line is the VAD-treated group.

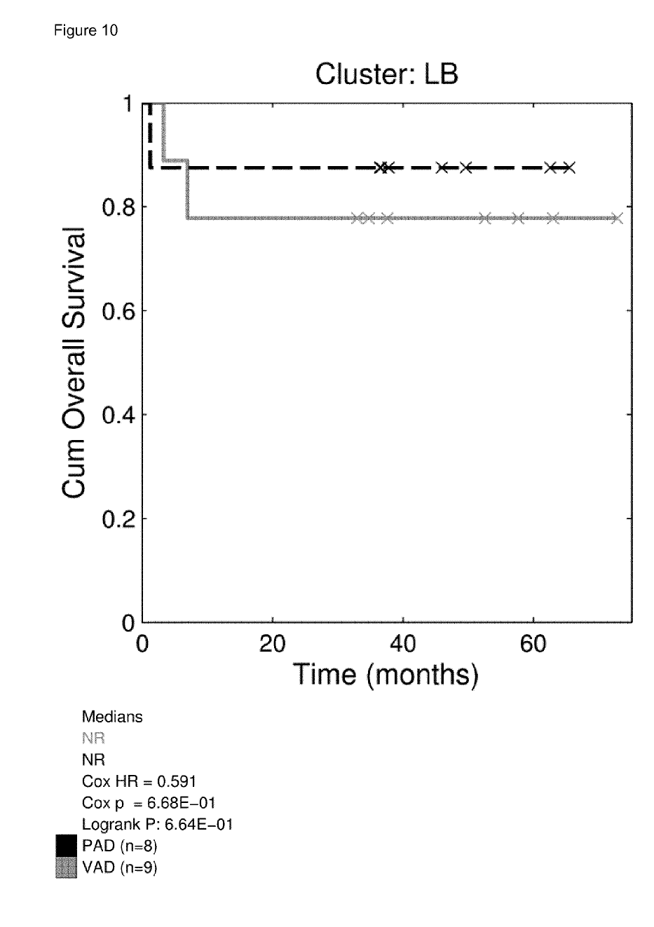

### DETAILED DESCRIPTION

### Examples

### Example 1: Study Design

A total number of 833 patients were included in a large prospective, randomized, phase III trial (HOVON-65/GMMG-HD4). Patients were randomly assigned to three cycles of induction treatment with vincristine, doxorubicin, and dexamethasone (VAD), or Bortezomib, doxorubicin, and dexamethasone (PAD). Both groups received high-dose melphalan with autologous stem-cell rescue followed by maintenance treatment with thalidomide (group assigned to VAD) or Bortezomib (group assigned to PAD) for 2 years (Sonneveld et al., J. Clin. Oncol. Vol. 30, 24:2946-2955, 2012).

The Ethics Committees of the Erasmus University MC, the University of Heidelberg and the participating sites approved this study. Informed consent to treatment protocols and sample procurement was obtained for all cases included in this study, in accordance with the Declaration of Helsinki. The institutional review board, ethics committee, of Erasmus MC approved use of diagnostic tumor material.

### Example 2: Gene Expression Profiling, Assessment of Outcome and Statistical Analysis

The gene expression dataset GSE19784 was used, derived from patients included in the HOVON-65/GMMG-HD4 trial (Broyl et al., Blood 2010 116:2543-2553). A total number of 320 patients were included in the molecular classification and follow-up data were available for 319 patients. Clusters with less than ten patients were not included in this study, the total number of patients was, therefore, 301 (Table 1). Progression-free survival (PFS) was calculated from randomization until progression, relapse or death, whichever came first. Patients who received a non-myeloablative allogeneic stem cell transplantation (AlloSCT) were censored at the date of AlloSCT. Overall survival (OS) was measured from randomization until death from any cause. Patients alive at the date of last contact were censored. The median follow-up was 41 months. Survival analysis was performed using the SPSS software. Kaplan

Meier analysis was performed using the log rank test to assess for significance in survival time between clusters.

### Example 3: Clustering of Patient Groups

The published myeloma classification (EMC classification, Broyl et al., Blood 2010 116:2543-2553) consisted of ten main clusters including CD-1, CD-2, MS, PR, HY, MF, Myeloid, NF-κB, CTA, and PRL-3. The MF cluster could be further subdivided in an LB subcluster, and an MF subcluster. In addition, one cluster did not have a clear gene expression signature, i.e., no profile (NP) cluster (Broyl et al., Blood 2010 116:2543-2553).

In the study described herein, the clusters PRL-3 and NP were disregarded since they consisted of less than ten patients. The LB and MF subclusters as identified in Broyl et al., Blood 2010, are considered as clusters herein.

### Example 4: Refined Method for Classifying Multiple Myeloma (MM) Patients into Clusters MS, MF, CD-1, NF-κB, or LB

This method employs array technology, for example, the Affymetrix Human Genome U133 Plus 2.0 microarray chip to measure mRNA levels of genes related to the clusters MS, MF, CD-1, NF-κB, and LB. Chip measurements were normalized using the MASS algorithm (trimmed mean scaled to 1500), log 2 transformed, followed by mean variance normalization per probeset.

Subsequently, for each of the clusters, a nearest centroid classifier was derived from the HOVON-65/GMMG-HD4 cohort of 329 samples using a double-loop cross-validation procedure. In the inner loop, learning curves were constructed to assess the accuracy across a range of 1 up to 100 probesets. These classifiers consider one cluster vs all other patients. For an MM patient x, a distance d to each of the two centroids was calculated, named Cluster and non-Cluster (e.g., MF and non-MF), using the normalized Euclidean distance measure. This results in a d_{Cluster} and a d_{non-Cluster}, formulated as:

$$\begin{matrix}{d}_{Cluster} ( x ) = \sqrt{{\sum }_{i = 1}^{N} \frac{{( {x}_{i} - {m}_{Cluster , i} )}^{2}}{{s}_{Cluster , i}^{2}} ,} & ( formula 1 ) \\ {d}_{non - Cluster} ( x ) = \sqrt{{\sum }_{i = 1}^{N} \frac{{( {x}_{i} - {m}_{non - Cluster , i} )}^{2}}{{s}_{non - Cluster , i}^{2}} ,} & ( formula 2 )\end{matrix}$$

where x indicates the expression levels of an MM patient to be classified, N is the total number of probesets used in the particular classifier, mᵢ the mean of the centroid for probeset i, and sᵢ the standard deviation of the centroid for probeset i. The MM patient is then assigned to the group with the smallest distance d (i.e. the closest centroid).

For example, considering the MF cluster and the first two genes in Table 11, the expression of these two genes is measured in a given patient. Next, the similarity with the MF and non-MF reference group is determined. A patient is then classified to the most similar group.

Learning curves indicated that each of the classifiers was highly accurate across the entire range of probesets. Probesets and centroids (means and standard deviations) used are listed in Tables 5 to 9 for the MS, MF, CD-1, NF-κB, and LB clusters, respectively.

The complete top 100 probeset ID lists are provided in Table 10. Subsets perform almost equivalently with the best performance when using the subsets indicated in Tables 5 to 9.

TABLE 5
Probesets and centroids of the MS cluster and non-MS cluster.

| | | Non-MS | | MS | |
|---|---|---|---|---|---|
| i | Probeset | mean (m) | sd (s) | mean (m) | sd (s) |
| 1 | 222777_s_at | −0.239 | 0.712 | 2.147 | 0.565 |
| 2 | 222778_s_at | −0.228 | 0.705 | 2.120 | 0.661 |
| 3 | 217867_x_at | −0.175 | 0.879 | 1.610 | 0.393 |
| 4 | 227084_at | −0.185 | 0.880 | 1.537 | 0.498 |
| 5 | 223472_at | −0.181 | 0.846 | 1.655 | 0.632 |
| 6 | 212771_at | −0.152 | 0.941 | 1.382 | 0.306 |
| 7 | 238116_at | −0.183 | 0.846 | 1.654 | 0.694 |
| 8 | 214156_at | −0.193 | 0.893 | 1.490 | 0.544 |
| 9 | 217901_at | −0.188 | 0.881 | 1.543 | 0.615 |
| 10 | 212686_at | −0.165 | 0.927 | 1.358 | 0.410 |
| 11 | 211709_s_at | −0.183 | 0.879 | 1.524 | 0.638 |
| 12 | 205559_s_at | −0.165 | 0.891 | 1.471 | 0.572 |
| 13 | 204066_s_at | −0.152 | 0.925 | 1.359 | 0.453 |
| 14 | 222258_s_at | −0.166 | 0.923 | 1.384 | 0.516 |
| 15 | 1557780_at | −0.184 | 0.840 | 1.607 | 0.826 |
| 16 | 223822_at | −0.189 | 0.833 | 1.590 | 0.823 |
| 17 | 1553105_s_at | −0.179 | 0.864 | 1.575 | 0.792 |
| 18 | 227692_at | −0.162 | 0.891 | 1.469 | 0.659 |
| 19 | 204379_s_at | −0.233 | 0.657 | 1.899 | 1.376 |
| 20 | 212190_at | −0.167 | 0.897 | 1.437 | 0.646 |

TABLE 6
Probeset IDs and centroids of the MF cluster and non-MF cluster.

| | | | Non-MF | | MF | |
|---|---|---|---|---|---|---|
| i | Probeset ID | Gene name | mean (m) | sd (s) | mean (m) | sd (s) |
| 1 | 204589_at | NUAK1 | −0.163 | 0.778 | 2.678 | 0.851 |
| 2 | 205718_at | ITGB7 | −0.094 | 0.911 | 1.903 | 0.512 |
| 3 | 221648_s_at | AGMAT | −0.103 | 0.914 | 1.723 | 0.471 |
| 4 | 205286_at | TFAP2C | −0.113 | 0.870 | 2.143 | 0.894 |
| 5 | 235228_at | CCDC85A | 0.086 | 0.937 | −1.643 | 0.465 |
| 6 | 222930_s_at | AGMAT | −0.086 | 0.937 | 1.621 | 0.470 |
| 7 | 1555756_a_at | CLEC7A | −0.123 | 0.878 | 1.897 | 0.789 |
| 8 | 1554406_a_at | CLEC7A | −0.127 | 0.868 | 1.936 | 0.939 |
| 9 | 1554485_s_at | TMEM37 | −0.084 | 0.936 | 1.707 | 0.650 |
| 10 | 204040_at | RNF144A | 0.103 | 0.890 | −1.952 | 0.953 |
| 11 | 226702_at | CMPK2 | 0.116 | 0.882 | −1.985 | 1.018 |

TABLE 7
Probeset IDs and centroids of the
CD-1 cluster and non-CD-1 cluster.

| | | Non-CD-1 | | CD-1 | |
|---|---|---|---|---|---|
| i | Probeset ID | mean (m) | sd (s) | mean (m) | sd (s) |
| 1 | 1555291_at | 0.028 | 0.957 | −1.288 | 0.792 |
| 2 | 213036_x_at | 0.079 | 0.930 | −1.452 | 1.333 |
| 3 | 205031_at | 0.012 | 0.973 | −0.997 | 0.622 |
| 4 | 207522_s_at | 0.064 | 0.962 | −1.216 | 1.125 |
| 5 | 212372_at | 0.025 | 1.008 | −0.832 | 0.411 |
| 6 | 231255_at | 0.020 | 0.979 | −1.031 | 0.813 |
| 7 | 210684_s_at | 0.018 | 0.983 | −0.939 | 0.691 |
| 8 | 1554625_at | 0.041 | 0.955 | −1.171 | 1.154 |
| 9 | 214840_at | 0.009 | 0.975 | −0.984 | 0.779 |
| 10 | 238931_at | 0.043 | 0.998 | −0.877 | 0.647 |
| 11 | 1558719_s_at | 0.042 | 0.898 | −1.167 | 1.255 |
| 12 | 213155_at | 0.024 | 1.011 | −0.749 | 0.386 |
| 13 | 228743_at | 0.037 | 0.980 | −1.024 | 0.928 |
| 14 | 235838_at | 0.031 | 0.995 | −0.879 | 0.657 |
| 15 | 207389_at | 0.022 | 0.985 | −0.960 | 0.808 |
| 16 | 240576_at | 0.035 | 0.969 | −1.021 | 0.965 |
| 17 | 1562256_at | 0.036 | 0.962 | −1.080 | 1.082 |
| 18 | 229452_at | 0.037 | 0.985 | −0.964 | 0.879 |
| 19 | 214694_at | 0.058 | 0.970 | −0.964 | 0.971 |
| 20 | 210872_x_at | 0.035 | 0.990 | −0.854 | 0.711 |
| 21 | 237206_at | 0.021 | 0.971 | −0.926 | 0.862 |
| 22 | 221413_at | 0.020 | 0.976 | −0.972 | 0.955 |
| 23 | 1557986_s_at | 0.006 | 0.999 | −0.738 | 0.497 |
| 24 | 1562495_at | 0.030 | 0.997 | −0.792 | 0.663 |

TABLE 8
Probeset IDs and centroids of the NF-κB cluster
and non-NF-κB cluster.

| | | Non-NF-κB | | NF-κB | |
|---|---|---|---|---|---|
| i | Probeset ID | mean (m) | sd (s) | mean (m) | sd (s) |
| 1 | 224783_at | −0.199 | 0.825 | 1.570 | 0.656 |
| 2 | 221970_s_at | 0.187 | 0.894 | −1.346 | 0.532 |
| 3 | 211444_at | −0.208 | 0.828 | 1.493 | 0.811 |
| 4 | 218715_at | 0.231 | 0.771 | −1.612 | 1.015 |
| 5 | 219146_at | 0.223 | 0.789 | −1.572 | 0.971 |
| 6 | 218014_at | 0.242 | 0.657 | −1.518 | 1.169 |
| 7 | 223780_s_at | −0.198 | 0.853 | 1.429 | 0.858 |
| 8 | 212130_x_at | −0.170 | 0.882 | 1.284 | 0.704 |
| 9 | 212227_x_at | −0.167 | 0.897 | 1.258 | 0.660 |
| 10 | 202630_at | 0.208 | 0.839 | −1.350 | 0.865 |
| 11 | 202631_s_at | 0.207 | 0.832 | −1.399 | 0.942 |
| 12 | 240126_x_at | −0.164 | 0.897 | 1.151 | 0.643 |
| 13 | 231853_at | 0.199 | 0.815 | −1.361 | 1.015 |
| 14 | 200614_at | 0.218 | 0.764 | −1.407 | 1.146 |
| 15 | 202021_x_at | −0.151 | 0.930 | 1.136 | 0.591 |
| 16 | 209600_s_at | 0.169 | 0.871 | −1.306 | 0.921 |
| 17 | 230012_at | 0.171 | 0.868 | −1.304 | 0.933 |
| 18 | 217672_x_at | −0.156 | 0.903 | 1.172 | 0.769 |
| 19 | 214696_at | −0.172 | 0.877 | 1.267 | 0.955 |
| 20 | 208863_s_at | 0.177 | 0.845 | −1.237 | 0.955 |
| 21 | 204760_s_at | −0.139 | 0.931 | 1.149 | 0.716 |

TABLE 9
Probesets and centroids of the LB cluster and non-LB cluster.

| | |
|---|---|
| Non-LB | LB |

| i | Probeset ID | mean (m) | sd (s) | mean (m) | sd (s) |
|---|---|---|---|---|---|
| 1 | 227949_at | −0.122 | 0.874 | 1.870 | 0.946 |
| 2 | 205590_at | −0.086 | 0.961 | 1.450 | 0.532 |
| 3 | 219895_at | 0.078 | 0.982 | −1.320 | 0.415 |
| 4 | 211986_at | −0.066 | 0.963 | 1.510 | 0.676 |
| 5 | 226702_at | 0.067 | 0.979 | −1.275 | 0.650 |

TABLE 10
Top 100 of genes of all clusters indicated by Probeset ID.

| i | MS | MF | CD-1 | NF-κB | LB |
|---|---|---|---|---|---|
| 1 | 222777_s_at | 204589_at | 1555291_at | 224783_at | 227949_at |
| 2 | 222778_s_at | 205718_at | 213036_x_at | 221970_s_at | 205590_at |
| 3 | 217867_x_at | 221648_s_at | 205031_at | 211444_at | 219895_at |
| 4 | 227084_at | 205286_at | 207522_s_at | 218715_at | 211986_at |
| 5 | 223472_at | 235228_at | 212372_at | 219146_at | 226702_at |
| 6 | 212771_at | 222930_s_at | 231255_at | 218014_at | 220850_at |
| 7 | 238116_at | 1555756_a_at | 210684_s_at | 223780_s_at | 205098_at |
| 8 | 214156_at | 1554406_a_at | 1554625_at | 212130_x_at | 200923_at |
| 9 | 217901_at | 1554485_s_at | 214840_at | 212227_x_at | 205159_at |
| 10 | 212686_at | 204040_at | 238931_at | 202630_at | 1564154_at |
| 11 | 211709_s_at | 226702_at | 1558719_s_at | 202631_s_at | 242625_at |
| 12 | 205559_s_at | 200951_s_at | 213155_at | 240126_x_at | 200989_at |
| 13 | 204066_s_at | 225868_at | 228743_at | 231853_at | 231963_at |
| 14 | 222258_s_at | 1554474_a_at | 235838_at | 200614_at | 213793_s_at |
| 15 | 1557780_at | 209708_at | 207389_at | 202021_x_at | 202145_at |
| 16 | 223822_at | 200953_s_at | 240576_at | 209600_s_at | 222281_s_at |
| 17 | 1553105_s_at | 1570445_a_at | 1562256_at | 230012_at | 213797_at |
| 18 | 227692_at | 224970_at | 229452_at | 217672_x_at | 202391_at |
| 19 | 204379_s_at | 211518_s_at | 214694_at | 214696_at | 225214_at |
| 20 | 212190_at | 210644_s_at | 210872_x_at | 208863_s_at | 219229_at |
| 21 | 212813_at | 242100_at | 237206_at | 204760_s_at | 244780_at |
| 22 | 212151_at | 213138_at | 221413_at | 227558_at | 206950_at |
| 23 | 212148_at | 241893_at | 1557986_s_at | 203967_at | 226560_at |
| 24 | 205830_at | 208373_s_at | 1562495_at | 202629_at | 226550_at |
| 25 | 201387_s_at | 224975_at | 220288_at | 242832_at | 227036_at |
| 26 | 238067_at | 221698_s_at | 227361_at | 221744_at | 213566_at |
| 27 | 217963_s_at | 210762_s_at | 235731_at | 229106_at | 224503_s_at |
| 28 | 41220_at | 209083_at | 232272_at | 215498_s_at | 228949_at |
| 29 | 213484_at | 231259_s_at | 1557569_at | 213021_at | 227367_at |
| 30 | 205131_x_at | 205862_at | 218030_at | 236668_at | 228274_at |
| 31 | 206045_s_at | 226806_s_at | 221464_at | 204640_s_at | 204422_s_at |
| 32 | 227290_at | 242625_at | 202192_s_at | 207667_s_at | 240405_at |
| 33 | 227372_s_at | 200762_at | 205873_at | 205811_at | 226651_at |
| 34 | 222738_at | 33323_r_at | 227271_at | 205527_s_at | 222833_at |
| 35 | 239297_at | 202688_at | 1557399_at | 209092_s_at | 222810_s_at |
| 36 | 226066_at | 210461_s_at | 44563_at | 200603_at | 204602_at |
| 37 | 222446_s_at | 226707_at | 239754_at | 224330_s_at | 209966_x_at |
| 38 | 241703_at | 229997_at | 242234_at | 226005_at | 240890_at |
| 39 | 218826_at | 229900_at | 209643_s_at | 200615_s_at | 204115_at |
| 40 | 200953_s_at | 211986_at | 1559682_at | 235089_at | 242785_at |
| 41 | 220991_s_at | 213737_x_at | 229175_at | 209076_s_at | 230499_at |
| 42 | 225530_at | 212724_at | 230076_at | 64438_at | 204567_s_at |
| 43 | 221261_x_at | 213093_at | 1558533_at | 226958_s_at | 221122_at |
| 44 | 214464_at | 229994_at | 34471_at | 211716_x_at | 229776_at |
| 45 | 200951_s_at | 237435_at | 41386_i_at | 221559_s_at | 209201_x_at |
| 46 | 224955_at | 228956_at | 1558875_at | 201742_x_at | 201843_s_at |
| 47 | 223313_s_at | 207638_at | 220566_at | 215499_at | 202688_at |
| 48 | 218775_s_at | 206020_at | 213067_at | 238923_at | 230389_at |
| 49 | 219631_at | 223866_at | 208005_at | 208927_at | 202687_s_at |
| 50 | 210220_at | 203417_at | 242832_at | 235728_at | 219024_at |
| 51 | 204749_at | 218935_at | 1553872_at | 201528_at | 226247_at |
| 52 | 220253_s_at | 204602_at | 219632_s_at | 205474_at | 202207_at |
| 53 | 219771_at | 202687_s_at | 236001_at | 203871_at | 204415_at |
| 54 | 205413_at | 205789_at | 1564360_a_at | 200816_s_at | 202011_at |
| 55 | 232235_at | 230740_at | 217348_x_at | 227159_at | 228450_at |
| 56 | 239246_at | 219895_at | 214805_at | 235609_at | 205801_s_at |
| 57 | 213155_at | 220234_at | 223460_at | 52169_at | 228115_at |
| 58 | 233437_at | 214639_s_at | 216964_at | 242938_s_at | 209030_s_at |
| 59 | 238605_at | 241048_at | 211495_x_at | 1554327_a_at | 229391_s_at |
| 60 | 205011_at | 220993_s_at | 208279_s_at | 218496_at | 219377_at |
| 61 | 209052_s_at | 218858_at | 1565723_at | 209849_s_at | 227889_at |
| 62 | 1556794_at | 1552618_at | 222844_s_at | 221326_s_at | 208358_s_at |
| 63 | 213940_s_at | 224822_at | 236006_s_at | 235688_s_at | 212724_at |
| 64 | 213012_at | 205898_at | 210314_x_at | 201518_at | 209309_at |
| 65 | 205560_at | 219370_at | 204592_at | 233936_s_at | 223823_at |
| 66 | 207233_s_at | 244461_at | 215232_at | 1554543_at | 216317_x_at |
| 67 | 204042_at | 228218_at | 210883_x_at | 223081_at | 210586_x_at |
| 68 | 203917_at | 219330_at | 223870_at | 202781_s_at | 221583_s_at |
| 69 | 201911_s_at | 209469_at | 238328_at | 242473_at | 242100_at |
| 70 | 208657_s_at | 219040_at | 217538_at | 213501_at | 222670_s_at |
| 71 | 204563_at | 226436_at | 230353_at | 225253_s_at | 237054_at |
| 72 | 204518_s_at | 203999_at | 226005_at | 222589_at | 203153_at |
| 73 | 218532_s_at | 49306_at | 228807_at | 217796_s_at | 239808_at |
| 74 | 209309_at | 1560316_s_at | 231068_at | 223259_at | 212158_at |
| 75 | 229874_x_at | 203304_at | 222779_s_at | 241910_x_at | 225589_at |
| 76 | 218258_at | 212067_s_at | 205951_at | 201168_x_at | 219355_at |
| 77 | 205120_s_at | 206167_s_at | 205527_s_at | 65493_at | 224341_x_at |
| 78 | 219440_at | 218723_s_at | 206995_x_at | 211095_at | 201842_s_at |
| 79 | 227367_at | 236760_at | 212713_at | 205094_at | 229390_at |
| 80 | 219983_at | 51158_at | 238096_at | 212708_at | 203865_s_at |
| 81 | 217975_at | 227542_at | 219794_at | 200605_s_at | 1564310_a_at |
| 82 | 204517_at | 208358_s_at | 219985_at | 239198_at | 229552_at |
| 83 | 207717_s_at | 239832_at | 215114_at | 227077_at | 214329_x_at |
| 84 | 200602_at | 222943_at | 243825_at | 202054_s_at | 219429_at |
| 85 | 226374_at | 208322_s_at | 203437_at | 201714_at | 203221_at |
| 86 | 203559_s_at | 226545_at | 224507_s_at | 235745_at | 237435_at |
| 87 | 223253_at | 221880_s_at | 219318_x_at | 212723_at | 224952_at |
| 88 | 225698_at | 235494_at | 220347_at | 206587_at | 210538_s_at |
| 89 | 210783_x_at | 226625_at | 211067_s_at | 201746_at | 202934_at |
| 90 | 241255_at | 222108_at | 1555063_at | 201436_at | 235490_at |
| 91 | 200711_s_at | 214329_x_at | 203871_at | 241239_at | 221909_at |
| 92 | 223663_at | 202946_s_at | 239916_at | 200604_s_at | 1562403_a_at |
| 93 | 236565_s_at | 222921_s_at | 1560281_a_at | 206917_at | 206762_at |
| 94 | 225710_at | 213793_s_at | 229726_at | 224785_at | 235065_at |
| 95 | 223703_at | 200952_s_at | 213146_at | 219123_at | 219525_at |
| 96 | 215047_at | 219660_s_at | 203267_s_at | 1553047_at | 221802_s_at |
| 97 | 218901_at | 202308_at | 236007_at | 243880_at | 1553678_a_at |
| 98 | 229269_x_at | 206394_at | 226833_at | 1554114_s_at | 211434_s_at |
| 99 | 217466_x_at | 229492_at | 208806_at | 232155_at | 216517_at |
| 100 | 218675_at | 230958_s_at | 1552664_at | 202871_at | 219211_at |

TABLE 11
Preferred genes for expression analysis of the MF cluster and the
non-MF cluster.

| | | | Non-MF | | MF | |
|---|---|---|---|---|---|---|
| i | Probeset ID | Gene name | mean (m) | sd (s) | mean (m) | sd (s) |
| 1 | 204589_at | NUAK1 | −0.163 | 0.778 | 2.678 | 0.851 |
| 2 | 205718_at | ITGB7 | −0.094 | 0.911 | 1.903 | 0.512 |
| 3 | 221648_s_at | AGMAT | −0.103 | 0.914 | 1.723 | 0.471 |
| 4 | 205286_at | TFAP2C | −0.113 | 0.870 | 2.143 | 0.894 |
| 5 | 235228_at | CCDC85A | 0.086 | 0.937 | −1.643 | 0.465 |
| 6 | 1554406_a_at | CLEC7A | −0.127 | 0.868 | 1.936 | 0.939 |
| 7 | 1554485_s_at | TMEM37 | −0.084 | 0.936 | 1.707 | 0.650 |
| 8 | 204040_at | RNF144A | 0.103 | 0.890 | −1.952 | 0.953 |
| 9 | 226702_at | CMPK2 | 0.116 | 0.882 | −1.985 | 1.018 |

### REFERENCES

Greipp P. R., J. San Miguel, and B. G. Durie, et al. International staging system for multiple myeloma. J. Clin. Oncol. 2005 23:3412-3420.
Sonneveld P., I. Schmidt-Wolf, and B. van der Holt, et al. Bortezomib induction and maintenance treatment in patients with newly diagnosed multiple myeloma: results of the randomized phase 3 HOVON-65/GMMG-HD4 trial. J. Clin. Oncol. in press 2012.
Broyl A., D. Hose, and H. Lokhorst, et al. Gene expression profiling for molecular classification of multiple myeloma in newly diagnosed patients. Blood 2010 116:2543-2553.
Cusack J. C. Rationale for the treatment of solid tumors with the proteasome inhibitor Bortezomib. Cancer Treat. Rev. 2003 29 Suppl. 1:21-31.
Hideshima T., D. Chauhan, P. Richardson, et al. NF-kappa B as a therapeutic target in multiple myeloma. J. Biol. Chem. 2002 277:16639-16647.
Mulligan G., C. Mitsiades, and B. Bryant, et al. Gene expression profiling and correlation with outcome in clinical trials of the proteasome inhibitor Bortezomib. Blood 2007 109:3177-3188.

1. A method for determining whether a subject with multiple myeloma is likely to respond to a treatment with a proteasome inhibitor, wherein the method comprises the step of performing, on a sample from the subject, a gene expression analysis of a number of N genes selected from the group consisting of genes NUAK1, ITGB7, AGMAT, TFAP2C, CCDC85A, CLEC7A, TMEM37, RNF144A, and CMPK2, wherein N is at least 2 and wherein it is concluded that the subject is likely to respond to a treatment with a proteasome inhibitor in the case where at least two of the N genes are aberrantly expressed.

2. The method according to claim 1, wherein the step of performing a gene expression analysis on a sample from the subject comprises the steps of:
a. providing at least one probe for the detection of the expression level of N genes selected from the group consisting of the genes NUAK1, ITGB7, AGMAT, TFAP2C, CCDC85A, CLEC7A, TMEM37, RNF144A, and CMPK2,
b. contacting the probe with said sample, and
c. determining the expression level of at least two genes from the at least N genes.

3. The method according to claim 1, wherein N is at least 3, 4, 5, 6, 7, 8, or at least 9.

4. The method according to claim 1, wherein it is concluded that the subject is likely to respond to a treatment with a proteasome inhibitor in the case where between 2 and N genes are aberrantly expressed.

5. The method according to claim 1, wherein the gene expression analysis is selected from the group consisting of gene array analysis, sequencing of RNA, RNA-FISH, quantitative-PCR, Northern Blotting, Multiplex Ligation Dependent Probe Amplification, microarray gene expression profiling and PCR.

6. The method according to claim 5, wherein the gene expression analysis is performed on a gene expression chip.

7. The method according to claim 1, wherein the proteasome inhibitor is selected from the group consisting of Bortezomib, Carfilzomib, MLN9708, Delanzomib, Oprozomib, AM-114, Marizomib TMC-95A, Curcusone-D and PI-1840.

8. The method according to claim 7, wherein the proteasome inhibitor is Bortezomib.

9. The method according to claim 1, wherein the treatment additionally comprises the administration of drugs selected from the group consisting of melphalan, prednisone, doxorubicin, dexamethasone, immunomodulating drugs, monoclonal antibody type drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

10. The method according to claim 1, wherein the sample comprises plasma cells.

11. The method according to claim 1, wherein a classifier is used to determine whether a gene is aberrantly expressed.

12. The method according to claim 11, wherein the classifier is a linear classifier.

13. The method according to claim 12, wherein the linear classifier is a ClaNC (Classification to Nearest Centroids) classifier.

14. The method according to claim 13, wherein for a single subject x with multiple myeloma, a distance d₀ and d₁ is calculated, wherein d₀ and d₁ are defined by the formulas 1 and 2:
$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$
wherein xᵢ represents the expression level of a particular gene i of the subject x, wherein gene i is chosen from the group comprising nine genes according to Table 11, wherein N is the total number of genes selected from the group comprising nine genes according to Table 11, wherein m₀ and s₀ are values according to Table 11, wherein mᵢ is the mean of the centroid for gene i according to Table 11, and wherein sᵢ is the standard deviation of the centroid for gene i according to Table 11 and wherein it is concluded that the subject x is likely to respond to treatment with a proteasome inhibitor if the value for d₁ is less than the value for d₀ or wherein it is concluded that the subject x is likely not to respond to a treatment with a proteasome inhibitor if the value for d₀ is less than or equal to the value for d₁.

15. A method of treating a subject with multiple myeloma, the method comprising:
a) performing, on a sample from the subject, a gene expression analysis of a number of N genes selected from the group consisting of the genes NUAK1, ITGB7, AGMAT, TFAP2C, CCDC85A, CLEC7A, TMEM37, RNF144A, and CMPK2, wherein N is at least 2;
b) determining the aberrant expression of at least two genes from the at least N genes; and
c) administering to the subject having aberrant expression of the at least two genes a therapeutically effective dose of a proteasome inhibitor.

16. The method according to claim 15, wherein the proteasome inhibitor is selected from the group consisting of Bortezomib, Carfilzomib, MLN9708, Delanzomib, Oprozomib, AM-114, Marizomib, TMC-95A, Curcusone-D and PI-1840.

17. The method according to claim 16, wherein the proteasome inhibitor is Bortezomib.

18. The method according to claim 15, wherein the treatment additionally comprises administering to the subject one or more drugs selected from the group consisting of melphalan, prednisone, doxorubicin, dexamethasone, immunomodulating drugs, monoclonal antibody type drugs, kinesin spindle protein (KSP) inhibitors, tyrosine kinase inhibitors, HDAC inhibitors, BCL2-inhibitors, Cyclin-dependent kinase inhibitors, mTOR inhibitors, heat-shock protein inhibitors, Bruton's kinase inhibitors, Insulin-like growth factor inhibitors, RAS inhibitors, PARP-inhibitors and B-RAF inhibitors.

19.-23. (canceled)

24. A method for determining whether a subject x, diagnosed with multiple myeloma, belongs to the MF cluster, the method comprising:
a) performing on a sample from the subject a gene expression analysis on one or more genes according to Table 11, and
b) calculating the probability that the subject belongs to the MF cluster based on the values for d₀ and for d₁, wherein a distance d₀ and d₁ is calculated, wherein d₀ and d₁ are defined by the formulas 1 and 2:
$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Formula 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Formula 2\end{matrix}$$
wherein xᵢ represents the expression level of a particular gene i of the subject x, wherein gene i is chosen from the group comprising nine genes according to Table 11, wherein N is the total number of genes selected from the group comprising nine genes according to Table 11, wherein m₀ and s₀ are values according to Table 11, wherein mᵢ is the mean of the centroid for gene i according to Table 11, and wherein sᵢ is the standard deviation of the centroid for gene i according to Table 11 and wherein it is concluded that the subject is likely to respond to treatment with a proteasome inhibitor if the value for d₁ is less than the value for d₀ or wherein it is concluded that the subject x is likely to belong to the MF-cluster if the value for d₁ is less than the value for d₀ or that the subject x is likely not to belong to the MF-cluster if the value for d₀ is less than or equal to the value for d₁.

25. (canceled)
